(19) United States
(12) Patent Application Publication
(10) Pub. No.: US 2006/0127317 A1
(43) Pub. Date: Jun. 15, 2006

(54) Nasallly applicable pharmaceutical preparation and the production thereof

(76) Inventors: Ernst Hesse, Fieberbrunn (AT); Gerhard Hantich, Kitzbuhel (AT); wolfgang H. Nitschmann, St. Johann I. T. (AT); Helmut Scheidl, Fieberbrunn (AT)

Correspondence Address: LERNER GREENBERG STEMER LLP, P O BOX 2480, HOLLYWOOD, FL, 33022-2480, US

(21) Appl. No.: 10/530,969

(22) PCT Filed: Oct. 9, 2003
(86) PCT No.: PCT/AT03/00306

§ 371 Date: Oct. 13, 2005

(30) Foreign Application Priority Data
Oct. 10, 2002 (AT) A 1540/2002

(51) Int. Cl. A61L 9/04

(52) U.S. Cl. 424/45

(57) ABSTRACT

The invention relates to a novel pharmaceutical preparation which can be administered nasally and is based on an aqueous solution, emulsion or the like which comprises at least one mucosally absorbable and/or locally acting active pharmaceutical ingredient, at least one preservative formed by benzalkonium chloride alone or together with other preservative substances, at least one buffer which keeps the pH at 4 to 6, and in addition at least one osmotic agent and/or at least one wetting agent and which is characterized in that the preparation has a substantially improved ciliary tolerability owing to the fact that in the preparation a buffer based on malic acid is present instead of a buffer which has been employed to date in the pharmaceutical preparation and is based on citrate(s), phosphate(s) and/or acetate(s)—partly or completely replacing it (them)—while retaining the composition, concentration and amount ratios, intended in each case for the pharmaceutical preparation, of active ingredient(s), preservative(s), osmotic agent(s) and wetting agent(s). It further relates to a process for producing the preparation and to the use of a malic acid buffer in the preparation.

The present invention relates to liquid preserved pharmaceutical preparations for administering various active ingredients on or in or via the nose of a patient in the form of a solution, to the production thereof and to the use of a specific buffer system for and in said preparations.

A large number of medicaments which can be administered in particular in the form of solutions and/or emulsions into the nose of a patient exists, either in the form of nasal drops or, increasingly recently, especially in the form of nasal sprays. These pharmaceutical preparations which can be administered nasally can be used either for the treatment or for the prevention of disorders of the nose itself, or else are intended to lead to uptake of active ingredients into the bloodstream, so that they display an effect elsewhere in the body.

Representatives which should be mentioned of the first-mentioned group of medicaments which can be administered nasally are, in particular, agents or active ingredients against nasal catarrh, such as allergy remedies such as, for example, cromoglicic acid, or sympathomimetics such as, for example, xylometazoline, tetrazoline, oxymetazoline, naphazoline, phenylephrine.

One example of the second group of such medicaments is represented for instance by peptide preparations such as, for example, those having desmopressin as active ingredient, which is an effective agent for the treatment of diabetes insipidus. Most such nasal sprays or nasal drops which are intended as medicaments comprise, besides at least one active ingredient,

substances to adjust a particular osmotic pressure (e.g. NaCl) and/or wetting or surface-active substances (e.g. Cremophors), also

excipients to stabilize the active ingredient or to maintain a particular physiologically acceptable pH in the nose. To date, phosphate or phosphate/citrate or citrate buffers and, in some circumstances, also acetate buffers have been employed almost exclusively for this purpose. In addition, in most cases they contain

benzalkonium chloride as preservative.

Benzalkonium chloride, abbreviated to BAC, has been employed widely as effective antiseptic and preservative since its introduction in 1935. There are reports in the scientific literature that it is well tolerated on the skin and on mucous membranes because it has scarcely any irritant effect (H. P. T. Ammon, Arzneimittelneben-and-wechselwirkungen, chapter 69: 1211; 1991, Mutschler E., Arzneimittelwirkungen, Lehrbuch der Pharmakologie und Toxikologie, chapter 9: 642; 1997).

Benzalkonium chloride is therefore employed very widely in disinfectants for the mouth and throat and for wound irrigation and vaginal douching. Because of the good antimicrobial activity and the good tolerability, it is the most frequently employed preservative, which is employed in nasal sprays and eye drops in conjunction with a large number of active pharmaceutical ingredients.

Ciliated epithelium, which is the ciliary apparatus of the nasal mucosa which is extremely important for maintaining the physiological function of the nose, is considerably impaired, in some cases even irreversibly, by the preservative benzalkonium chloride (Klöcker and Rudolph, P Z 145 (21) 40-42, 2000; Hofmann, et al. HNO 1998; 46 (2): 146-151; Neugebauer et al., annual meeting of the German society for otorhinolaryngology, head and neck surgery, 1998.

Some authorities in the European Union have in fact for this reason proposed or required a massive restriction of the use of this preservative (German Federal Institute for drugs, graduated plan procedure II, Bundesanzeiger No. 120, Jul. 3, 2002), although nasal sprays with benzalkonium chloride as preservative are currently used to treat, besides nasal catarrhal and allergic disorders directly affecting the respiratory system, also numerous chronic disorders which are often in fact life-long.

Pharmaceutical preparations, which are entirely free of preservatives are demanded as the only remedy for these problems deriving from the adverse effect of BAC on the ciliary apparatus. However, these preparations are associated with considerable economic expenditure. It is necessary to employ special nasal spray systems, and, inter alia, all packaging materials and sprayer parts which come into contact with the nasal spray solution must be subjected to elaborate sterilization with highly toxic chemicals and/or radioactive beams, which is, after all, not precisely desired either.

During detailed investigations with the purpose of finding a feasible remedy for this, it has emerged in a perfectly surprising manner that it is possible by use of a very particular, specific buffer system—known per se as such—to eliminate at least a large part of the damaging effect on the cilia and their activity of BAC which is valued and approved as preservative in its other properties.

The present invention thus relates to a novel pharmaceutical preparation which can be administered nasally and is based on an aqueous solution, emulsion or the like which comprises at least one mucosally absorbable and/or locally acting active pharmaceutical ingredient known per se, at least one preservative formed by benzalkonium chloride alone or together with other preservative substances, at least one buffer which keeps the pH at 4 to 6 or at about 5, and in addition at least one osmotic agent and/or at least one wetting agent and which is characterized in that the preparation has a substantially improved ciliary tolerability owing to the fact that in the same solution, emulsion or the like, or in the one underlying it, a buffer based on malic acid is present instead of a buffer which has been employed to date in the pharmaceutical preparation and is based on citrate(s), phosphate(s) and/or acetate(s)—partly or completely replacing it (them)—while retaining the composition, concentration and amount ratios, intended in each case for the pharmaceutical preparation, of active ingredient(s), preservative(s), osmotic agent(s) and wetting agent(s).

The present invention is a case which is not so common in the field of pharmaceutical preparations, where the essence thereof consists not of a novel active ingredient and the use thereof in a medicament, but on the contrary in the apparently substantially less spectacular area of an additive which has long ago become routine and has long been approved in practice, such as precisely the buffer system which is present in a medicament preparation and is crucial for its activity and stability, and in an unexpectedly beneficial change away from approved and generally widely employed buffer systems for liquid pharmaceutical preparations towards another buffer which is used substantially less often in medicaments.

It is quite essential to emphasize that the advantage of the present invention is that the change from the previous buffer system to the malic acid buffer which is now to be employed can take place without an alteration in the amount, concentration, composition ratios of the other components including the active ingredients in the various medicament preparations approved in practice, while costly rearrangements and authority procedures can be avoided.

Concerning a previously disclosed use of malic acid buffers in compositions for pharmaceutical and possibly also diagnostic purposes, reference should be made for example to WO 98/47490 which relates to lyophilizates of biomolecules and in which mention is made, besides a large number of different buffers based on organic acids, also of malic acid, and although the buffers mentioned therein are used to adjust the pH, their main task is to prevent the formation of interfering arginine phosphate—or arginine citrate-protein aggregates produced when phosphate or citrate buffers known per se are used.

Mention should further be made, concerning the use of a malic acid buffer in a pharmaceutical preparation which can be used orally sublingually or nasally and comprises the active ingredient desmopressin, of our own AT 409 081 B1, according to which a substantial improvement in the stability of the pharmaceutical preparation can be achieved through the use of the malic acid buffer, whether together with substantially reduced amounts of benzalkonium chloride as preservative or with exclusion thereof.

Neither of the two publications mentioned touches even in the smallest degree on the problems solved by the present invention, of the damage mainly caused by the approved preservative benzalkonium chloride on the ciliary apparatus of the nasal mucosa, which is highly relevant especially when a medicament preparation which can be administered nasally must be administered over a long period.

The simplest and perfectly effective embodiment for the purpose of reducing the harmful effect on the cilia of this preservative and, in particular, also rapid and substantial regeneration of the ciliary activity after administration of the medicaments with a wide variety of medicament active ingredients comprising the same, as already emphasized above, is racemic malic acid as pH stabilizer and agent which precisely prevents very substantially the harmful effects on the cilia. However, enantiopure malic acid can also be employed instead of racemic malic acid.

Particularly appropriate concentration ranges of the malic acid buffer in the novel pharmaceutical preparation are indicated in Claim 2.

Malic acid buffers in which sodium hydroxide solution is employed to form the counter ion in the buffer system have proved appropriate, as is evident from claim 3.

Claim 4 names NaCl as a particularly appropriate osmotically active ingredient in connection with the cilia-friendly effect of the novel medicament preparations.

Claim 5 mentions, without claiming completeness, some active ingredient groups and specific important active ingredients which can be employed in the preparations of the invention with the malic acid buffer.

A further essential aspect of the present invention is formed by the process for producing the novel pharmaceutical preparation which can be administered nasally, details of which are mentioned in claim 6, the essential feature of this production process being the specific replacement of the buffer systems which have previously been employed—and have proved in the course of the investigations for the present invention to be—in the presence of benzalkonium chloride—thoroughly dangerous for ciliary activity, by a malic acid buffer.

The features of claims 7 and 8, which follow the production claim 6 and refer back to it, are analogous to the features of claims 2 to 6 already explained above.

A further essential aspect of the invention consists of the—as described above—surprisingly found use, which has not to date been mentioned or even suggested anywhere, of the buffer system based on malic acid as essential ingredient, instead of buffers customary to date for producing cilia-tolerated pharmaceutical preparations which can be administered nasally, the details not being quoted here and being disclosed in claim 9.

The features of claims 10 and 11, which follow the use claim 9 and refer to it and likewise refer to the use of a malic acid buffer in the medicament preparations under consideration, are analogous to those of claims 2 to 4 and 7 and 8 explained in detail above.

Finally, reference should also be made to the further aspect of the invention relating to the use of the malic acid buffer system in the novel pharmaceutical preparations, which is to be found in its entirety in claim 12.

The invention is explained in more detail by means of the following examples.

### GENERAL EXAMPLE

In a standard test for examining ciliary function, a conventional preparation for nasal administration having a phosphate/citrate buffer and benzalkonium chloride as preservative was compared in each case with a preparation of the invention having malic acid as buffer and benzalkonium chloride as preservative.

Pieces of tissue from ciliated epithelium of the trachea of chicken embryos are employed in the tests on which the comparison is based, reference being made for details of the tests to S. G. Romejn et al., Int. J. of Pharmac. 135 (1996) 137-145 and van De Donk et al., Rhinology, 20 (1982) 81-87.

Active ingredient solution 1

0.335 mg/ml malic acid; 0.168 mg/ml NaOH; 9.115 mg/ml sodium chloride; 0.1 mg/ml benzalkonium chloride; pH: 5.07.

Active ingredient solution 2

1.7 mg/ml citric acid; 3 mg disodium phosphate dihydrate; 7.5 mg/ml sodium chloride; 0.1 mg/ml benzalkonium chloride; pH: 5.04.

| Preparation | Ciliary frequency | Reversibility of |
|---|---|---|
| with | after incubation | the effect in |
| desmopressin | for 15 min, % of | Ringer's solution |
| as active | the original | (45 min), % of the |
| ingredient | frequency; | original frequency; |
| (n = 6) | standard deviation | standard deviation |
| | in brackets | in brackets |
| Solution 1 with | 33 (13) | 74 (15) |
| malic acid | | |
| buffer | | |
| Solution 2 with | 6 (7) | 48 (18) |
| normal buffer | | |
| Control with | 101 (6) | 102 (3) |
| Ringer's | | |
| solution | | |

After exposure for 15 min, the ciliary frequency is reduced distinctly less by solution 1 with malic acid buffer than by solution 2 with the usual buffer. The self-cleaning of the nasal mucosa is then simulated by washing out the respective solutions and adding Ringer's solution for 45 min. After this, a distinctly increased, from 48 to 74%, recovery of the ciliary frequency is achieved with solution 1 with the malic acid buffer.

### Example 1

4900 g of distilled water are introduced into a 5 l glass beaker and 45.58 g of sodium chloride, 0.5 g of benzalkonium chloride, 1.675 g of malic acid and 5 g of xylometazoline hydrochloride are dissolved therein by stirring. The pH is adjusted to 5.5 with 1N NaOH. The volume is made up to 5 l with distilled water, and the resulting solution is further processed to nasal drops or to a nasal spray.

### Example 2

4900 g of distilled water are introduced into a 5 l glass beaker and 45.58 g of sodium chloride, 0.5 g of benzalkonium chloride, 1.675 g of malic acid and 2.5 g of xylometazoline hydrochloride are dissolved therein by stirring. The pH is adjusted to 5.5 with 1N NaOH. The volume is made up to 5 l with distilled water, and the resulting solution is further processed to nasal drops or to a nasal spray.

### Example 3

4900 g of distilled water are introduced into a 5 l glass beaker and 45.58 g of sodium chloride, 0.5 g of benzalkonium chloride, 1.675 g of malic acid and 12.5 g of phenylephrine hydrochloride are dissolved therein by stirring. The pH is adjusted to 5 with 1N NaOH. The volume is made up to 5 l with distilled water, and the resulting solution is further processed to nasal drops or to a nasal spray.

### Example 4

4900 g of distilled water are introduced into a 5 l glass beaker and 45.58 g of sodium chloride, 0.5 g of benzalkonium chloride, 0.67 g of malic acid and 6.25 g of phenylephrine hydrochloride are dissolved therein by stirring. The pH is adjusted to 5 with 1N NaOH. The volume is made up to 5 l with distilled water, and the resulting solution is further processed to nasal drops or to a nasal spray.

Summary of the results achieved with the preparations according to examples 1 to 4 in relation to the substantial improvement in ciliary tolerability.

The composition of the reference solutions for examples 1 to 4 is shown in table 1 below:

TABLE 1

| | Reference | | Reference | | Reference | | Reference | |
|---|---|---|---|---|---|---|---|---|
| | 1 | | 2 | | 3 | | 4 | |
| Xylometazoline | 1 | mg | 0.5 | mg | | | | |
| hydrochloride | | | | | | | | |
| Phenylephrine | | | | | 2.5 | mg | 1.25 | mg |
| base | | | | | | | | |
| Sodium | 5 | mg | 5 | mg | | | | |
| dihydrogenph. | | | | | | | | |
| dihydrate | | | | | | | | |
| Sodium mono- | | | | | | | | |
| hydrogenphosphate | 1.7 | mg | 1.7 | mg | | | | |
| dodecahydrate | | | | | | | | |
| Disodium | | | | | 4.6 | mg | 2.3 | mg |
| hydrogenphosphate | | | | | | | | |
| Citric acid. H₂O | | | | | 2.6 | mg | 1.3 | mg |
| Disodium edetate | 0.45 | mg | 0.45 | mg | | | | |
| Benzalkonium | 0.1 | mg | 0.1 | mg | 0.1 | mg | 0.1 | mg |
| chloride | | | | | | | | |
| Sorbitol | 21 | mg | 21 | mg | 50 | mg | 60 | mg |
| Sodium chloride | 5 | mg | 5 | mg | | | | |

| | | | | |
|---|---|---|---|---|
| Water | ad 1 ml | ad 1 ml | ad 1 ml | ad 1 ml |

The comparative results relating to ciliary tolerability are summarized in table 2.

TABLE 2

| | Ciliary frequency | Reversibility of the |
|---|---|---|
| | after incubation for | effect in Ringer's |
| | 15 min, % of the | solution (45 min), % |
| | original frequency; | of the original |
| | standard deviation in | frequency; standard |
| Preparation | brackets | deviation in brackets |
| Solution of | 29 (16) | 71 (16) |
| example 1 | | |
| Reference | 12 (5) | 36 (15) |
| solution 1 | | |
| Solution of | 34 (12) | 79 (19) |
| example 2 | | |
| Reference | 18 (6) | 41 (11) |
| solution 2 | | |
| Solution of | 36 (11) | 68 (18) |
| example 3 | | |
| Reference | 17 (7) | 29 (13) |
| solution 3 | | |
| Solution of | 38 (14) | 77 (16) |
| example 4 | | |
| Reference | 15 (8) | 40 (14) |
| solution 4 | | |

After exposure for 15 min, the ciliary frequency is reduced distinctly less by the exemplary solutions with malic acid buffer than by the reference solutions. The self-cleaning of the nasal mucosa is then simulated by washing out the test solutions and adding Ringer's solution for 45 min. A distinctly better recovery of the ciliary beating force, which extends to more than ¾ of the initial beating force (of 100%), is achieved thereby with the exemplary solutions with malic acid buffer. This value is very good, especially since only 55% of the initial ciliary frequency are obtained after 45 mins even on incubation of the cilia in physiological saline solution.

### Example 5

A nasal spray with benzalkonium chloride as preservative for treating diuretic impairments and bleeding disorders is produced by introducing 990 g of water for injections into a 1 l glass beaker and dissolving therein 9.115 g of sodium chloride, 0.1 g of desmopressin acetate, 0.1 g of benzalkonium chloride and 0.335 g of malic acid. The pH is adjusted to 5 with 4.2 ml of 1N NaOH, and then the volume is made up to 1 l, and the solution is filtered through a Millipak filter and dispensed in amber glass bottles which are closed with pump attachments. The production and dispensing of the solution take place in pharmaceutical manufacturing rooms under low-microbe conditions.

The results obtained in orienting tests with the active ingredient solution of example 5 were quite analogous to the results listed in table 2 above in terms of substantially less reduction in ciliary activity and improved regeneration of the cilia.

The results obtained in further, likewise orienting tests with the active ingredients calcitonin (from salmon) for the treatment of osteoporosis and cromoglicic acid for the treatment of allergic nasal catarrh were similar.

1-16. (canceled)

17. A buffered pharmaceutical preparation for nasal administration, the preparation comprising:
water;
at least one nasally administrable active pharmaceutical ingredient;
at least one preservative comprising benzalkonium chloride;
at least one buffer keeping the pH at 4 to 6, said at least one buffer comprising a malic acid compound; and
at least one agent selected from the group consisting of an osmotic agent and a wetting agent;
said preparation having substantially improved ciliary tolerability.

18. The preparation according to claim 17, wherein said malic acid compound is present in a concentration in a range from 1 to 5 millimoles per liter of said pharmaceutical preparation.

19. The preparation according to claim 17, wherein said buffer is formed with sodium as counter ion.

20. The preparation according to claim 17, wherein said malic acid compound is selected from the group consisting of racemic malic acid and enantiopure malic acid.

21. The preparation according to claim 17, wherein said osmotic agent comprises sodium chloride.

22. The preparation according to claim 17, wherein said active pharmaceutical ingredient is selected from the group consisting of at least one allergy remedy, at least one sympathomimetic remedy, at least one nasal catarrh remedy, at least one corticoid, at least one peptide, and at least one hormone.

23. The preparation according to claim 17, wherein said active pharmaceutical ingredient is selected from the group consisting of levocabastine, azelastine, cromoglicic acid, xylometazoline, tetrazoline, indanazoline, phenylephrine, naphazoline, tramazoline, oxymetazoline, beclometasone, triamcinolone, calcitonin, desmopressin, gonadorelin, buserelin, nafarelin, and oxytocin.

24. The preparation according to claim 17, wherein said active pharmaceutical ingredient is calcitonin.

25. The preparation according to claim 17, wherein said active pharmaceutical ingredient is cromoglycic acid.

26. The preparation according to claim 17, wherein said active pharmaceutical ingredient is desmopressin.

27. The preparation according to claim 17, wherein said active pharmaceutical ingredient is phenylephrine.

28. The preparation according to claim 17, wherein said active pharmaceutical ingredient is xylometazoline.

29. The preparation according to claim 17, additionally comprising a buffer selected from the group consisting of a citrate buffer, a phosphate buffer, and an acetate buffer.

30. The preparation according to claim 17, being an emulsion.

31. The preparation according to claim 17, being a solution.

32. The preparation according to claim 17, wherein said active pharmaceutical ingredient is mucosally absorbable.

33. The preparation according to claim 17, wherein said active pharmaceutical ingredient is locally effective.

34. A method of treating a condition selected from the group consisting of allergy, bleeding disorder, diuretic impairment, and osteoporosis, comprising the intranasal administration to a person in need of such treatment of the preparation according to claim 17.

35. The method of claim 34, which comprises treating the person by administering the preparation via a nasal spray.

36. The method of claim 34, which comprises treating the person by administering the preparation via nose drops.

37. A method of preparing a buffered pharmaceutical preparation for nasal administration and having substantially improved ciliary tolerability, the method which comprises:
providing water;
admixing at least one nasally administrable active pharmaceutical ingredient;
admixing at least one preservative comprising benzalkonium chloride and at least one agent selected from the group consisting of an osmotic agent and a wetting agent; and buffering the preparation to a pH of 4 to 6 with at least one buffer at least primarily comprising a malic acid compound.
